(19) United States
(12) Patent Application Publication
(10) Pub. No.: US 2014/0180376 A1
(43) Pub. Date: Jun. 26, 2014

(54) INTRINSIC TRANSDUCTION SYSTEM

(71) Applicant: JAMES EDWARD JENNINGS, Superior, CO (US)

(72) Inventor: JAMES EDWARD JENNINGS, Superior, CO (US)

(21) Appl. No.: 13/724,287

(22) Filed: Dec. 21, 2012

(51) Int. Cl. A61N 1/04

(52) U.S. Cl. CPC A61N 1/0456
USPC 607/144; 29/601

(57) ABSTRACT

An intrinsic transduction system for stimulating portions of the body. The output of the transducer is a stimulating and/or nerve regenerating frequency having components falling within predetermined frequency band limits so as to optimally excite touch nerve fibers relative to nociceptor or pain receptor nerve fibers. Products, in particular a prosthetic disc, shoe, apparel, a spark cleat, incorporating an impact sensing element made from polymeric piezoelectric material. In response to impact, the piezoelectric material generates an electrical signal to a battery powered negative ion emitting unit or to an information display device which is at least partially molded into or contained in the product, or relayed by antenna. In addition, a CPU can be included in the circuitry to provide preprogrammed control of the emitting devices or to evaluate the input from the impact sensing element and a negative ion transducer, permits ready control of a quantity of generated negative ions and resists a size and thickness reduction. The negative ion transducer is of an electron emission type in which electrons are emitted by spin oscillation and/or impressing a negative ion voltage electric discharge . A negative ion transistor chip is used for amplifying a voltage from a capacitor and circuit.

### BACKGROUND OF THE INVENTION

1. Field of the Invention

This invention relates to transduction piezoelectric nerve stimulation and more specifically relates to devices for applying transcutaneous nerve stimulation for physiotherapeutic purposes. The present disclosure relates generally to systems and methods for causing nerve cells to regenerate and, more particularly, to systems and methods for promoting nerve regeneration in the central and peripheral nervous stimuli systems of humans.

Transcutaneous nerve stimulation, commonly referred to as TENS is the application of a controlled amount of low electrical currents to stimulate nerves and/or muscle tissues in a patient for treating numerous physiological problems such as muscle and joint pain and inflammation. The currents may be provided in a steady flow or in electrical impulses of various wavelength frequencies. The electrical currents primarily stimulate the nerve for the body to produce natural endorphins to block the perception of pain and also physically cause the muscle tissues at the area of application to tighten and relax repeatedly, and thus increasing the blood circulation to enhance the natural curing process. The TENS currents are provided by a generator and the currents are delivered with application probes to the inflicted locations of a patient's body. The free end of the currents application probes is commonly in the form of a flexible inductive composite pad which must be attached to the patient's body with conductive adhesive gel and/or adhesive tapes in order to deliver the current to the patient's body. However, the curing process is not efficient if it is relying solely on the TENS stimulation.

Peripheral nerve fibers have been classified in order of decreasing size and conduction velocity in a manner which is now standardized. Generally, as the fibre size decreases, the amplitude of electrical stimulation required to elicit an action potential increases. Also, the smaller fibre will require longer pulse durations than large fibre stimuli. These differences in nerve response have been used to selectively stimulate different types of nerve fibers by varying the amplitude, pulse duration, or pulse repetition rate of an electrical stimulating pulse. The desired degree of nerve fibre selectivity, however, has not been achieved in the prior art, with the result that, for example, an elicited touch response resulting from the stimulating pulse is often accompanied by a prickly, stinging, burning, sharp or other unpleasant noxious response.

Therefore, various exemplary embodiments of the invention may provide a nerve regeneration system that may include an interactive diagnostic device configured to measure nerve growth, re-growth, and/or connections between severed or otherwise damaged nerve segments.

To attain the advantages and in accordance with the purpose of the invention, as embodied and broadly described herein, one exemplary aspect of the invention may provide a nerve regeneration system comprising a lead configured to be placed in a body proximate a damaged nerve, a portion of the lead being configured to stimulate the damaged nerve.

According to one exemplary aspect, the stimulation comprises a therapeutic electric signal, and the parameter of the stimulation may comprise a parameter associated with the electric signal. For example, the parameter may comprise one or more of strength, direction, current, or voltage of the electric signal. According to another exemplary aspect, the nerve regeneration system may comprise an electrode coupled to the lead and configured to deliver electric stimulation to the damaged nerve. The electrode may include a plurality of electrodes and the parameter may comprise one or more of a number, a sequence, or a combination of electrodes to be energized to deliver electric stimulation. The system may also comprise a conductor for connecting the electrode to the control module. According to still another aspect, the control module may be enclosed in a substantially sealed housing with one or more leads extending from the housing. The control module may be configured to communicate with an external device. According to another aspect, the present disclosure is directed toward a nerve regeneration system that comprises a nerve regeneration module comprising at least one lead implanted in a body proximate a damaged nerve. The nerve regeneration module may be configured to administer a nerve regeneration treatment to the damaged nerve and detect a patient response to the nerve regeneration treatment. According to still another aspect, the nerve regeneration system comprises a power supply configured to generate an electromagnetic signal for stimulating the damaged nerve. Accordingly, the at least one lead may comprise one or more electrodes electrically coupled to the power supply, the one or more electrodes being configured to deliver the electromagnetic signal to the damaged nerve. According to one embodiment, the one or more of the electrodes are disposed along a length of the at least one lead.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

2. Description of Related Art

The central nervous system, including the brain, is the primary control system of a body, communicating with one or more parts of the body via a complicated system of interconnected nerves. Nerves are cable-like bundles of axons that carry electrical signals and impulses between one or more neurons and the central nervous system. Thus, nerves play a critical role in communicating sensory and stimulatory signals between various parts of the body (e.g., muscles, organs, glands, etc.) and the central nervous system.

Nerves may be damaged or severed either through trauma or disease. Damaged or severed nerves may inhibit the central nervous system's ability to receive sensory and stimulatory data from individual neurons, potentially limiting the nervous system's control over the body. For example, severe nerve damage may lead to paralysis, such as paraplegia or quadriplegia.

In the peripheral nervous system, a common treatment to repair damaged nerves involves a surgical procedure to harvest a healthy nerve from another part of the patient's body and graft the harvested nerve to bridge the damaged section. Although surgery can successfully repair damaged nerve cells in many cases, these procedures may have several disadvantages. For instance, in most cases, several invasive surgical procedures are required to find suitable donor nerves. Further, damage to nerves at the donor site is quite common, potentially leading to weakening of donor nerves at the expense of the recipient nerves.

Some alternatives to surgical repair of damaged nerves have been developed. These systems typically involve surrounding damaged nerves in a sheath and administering therapeutic drugs or electromagnetic energy to the damaged nerve site. The administration of the therapeutic drugs and/or electromagnetic energy may facilitate nerve regeneration, while the sheath guides the nerve to grow in a desired direction.

Engineer Georges Lakhovsky, believed that people could achieve good health by adjusting the oscillation of their cells. He tapped Tesla to assist him in building the Multiple Wave Oscillator. Lakhovsky claimed the machine would improve health, remove pathogens, and even cure cancer. “The action of the pounding surf creates negative air ions and we also see it immediately after spring thunderstorms when people report lightened moods,” says ion researcher Michael Terman, PhD, of Columbia University in New York. The Organic Electronics research group at Linkoping University previously developed ion transistors for transport of both positive and negative ions, as well as biomolecules. An advantage of chemical circuits is that the charge carrier consists of chemical substances with various functions. This means that we now have new opportunities to control and regulate the signal paths of cells in the human body.

Energy in electronic elements: Electric potential energy, or electrostatic potential energy, is a potential energy (measured in joules) that results from conservative Coulomb forces and is associated with the configuration of a particular set of point charges within a defined system. The term “electric potential energy” is used to describe the potential energy in systems with time-variant electric fields, while the term “electrostatic potential energy” is used to describe the potential energy in systems with time-invariant electric fields.

Capacitance is the ability of a body to store an electrical charge. Any body or structure that is capable of being charged, either with static electricity or by an electric current, exhibits capacitance. A common form of energy storage device is a parallel-plate capacitor. In a parallel plate capacitor, capacitance is directly proportional to the surface area of the conductor plates and inversely proportional to the separation distance between the plates. If the charges on the plates are +q and −q, and V gives the voltage between the plates, then the capacitance C is given by

C=q/V.

The capacitance is a function only of the physical dimensions (geometry) of the conductors and the permittivity of the dielectric. It is independent of the potential difference between the conductors and the total charge on them. Piezoelectricity is the combined effect of the electrical behavior of the material:

D=εE

where D is the electric charge density displacement (electric displacement), ε is permittivity and E is electric field strength, and

### Hooke's Law: S=s T

where S is strain, s is compliance and T is stress.

### Physical Properties of TPU

TPU possesses a combination of physical properties not available in other thermoplastic materials or synthetic rubbers, including: Superior Abrasion resistance for physically punishing, high-wear applications. Formulated UV resistance prevents yellowing or embrittlement. Elevated tensile strength provides reliability and durability over the life of the product in which the film is used. Good memory retention, Durometers (hardness) from very soft to very hard. High resistance to hydrocarbons, chemicals, ozone, bacteria, and fungus make it ideal for tough industrial environments Inherently waterproof, for use in performance apparel, bedding, transdermal and wound care applications. Superior resistance to skin oils, yet has good “hand” or “feel” when in contact with the skin Easily fabricated using thermal bonding, laminating, die cutting, radio frequency (RF) sealing or vacuum forming and Flame-retardant. Typically, when two or more of these properties are required for an application, TPU is the material of choice.

### Other TPU Medical Applications

TPU is typically used for parts requiring a high level of performance. Applications typically require a flexible material with a high degree of flex resistance, wearability and durability. Many of the characteristics of TPU make it ideal for medical use. Medical applications include: IV site dressings, Transdermal patches, Thin film wound dressings, Cast and dressing covers, Surgical gowns & drapes, Puncture-resistant gloves, Incontinence pads, Compression dressings, Orthopedic gel insoles, Medical anti-shock trousers, Gel-filled positioning pads, Inflatable support bladders, Pressure infuser cuffs, Extraction bags, Hospital mattresses, covers, Orthodontic brace aligners.

Copolymers: Copolymers of PVDF are also used in piezoelectric and electrostrictive applications. One of the most commonly-used copolymers is P(VDF-trifluoroethylene), usually available in ratios of about 50:50 wt % and 65:35 wt % (equivalent to about 56:44 mol % and 70:30 mol %). Another one is P(VDF-tetrafluoroethylene). They improve the piezoelectric response by improving the crystallinity of the material.

A novel electrospun TPU/PVdF porous fibrous polymer electrolyte for lithium ion batteries. Novel blend-based gel polymer electrolyte (GPE) films of thermoplastic polyurethane (TPU) and poly(vinylidene fluoride) (PVdF) (denoted as TPU/PVdF) have been prepared by electrospinning The electrospun thermoplastic polyurethane-co-poly (vinylidene fluoride) membranes were activated with a 1M solution of LiClO4 in EC/PC and showed a high ionic conductivity about 1.6 mS cm−1 at room temperature. The electrochemical stability is at 5.0 V versus Li+/Li, making them suitable for practical applications in lithium cells. Cycling tests of Li/GPE/LiFePO4 cells showed the suitability of the electrospun membranes made of TPU/PVdF (80/20, w/w) for applications in lithium rechargeable batteries.

A novel high-performance gel polymer electrolyte membrane basing on electrospinning technique for lithium rechargeable batteries. Nonwoven films of composites of thermoplastic polyurethane (TPU) with different proportion of poly(vinylidene fluoride) (PVdF) (80, 50 and 20%, w/w) are prepared by electrospinning 9 wt % polymer solution at room temperature. Then the gel polymer electrolytes (GPEs) are prepared by soaking the electrospun TPU-PVdF blending membranes in 1 M LiC1O4/ethylene carbonate (EC)/propylene carbonate (PC) for 1 h. The gel polymer electrolyte (GPE) shows a maximum ionic conductivity of 3.2×10-3 S cm−1 at room temperature and electrochemical stability up to 5.0 V versus Li+/Li for the 50:50 blend ratio of TPU:PVdF system. At the first cycle, it shows a first charge—discharge capacity of 168.9 mAh g−1 when the gel polymer electrolyte (GPE) is evaluated in a Li/PE/lithium iron phosphate (LiFePO4) cell at 0.1 C-rate at 25° C. TPU-PVdF (50:50, w/w) based gel polymer electrolyte is observed much more suitable than the composite films with other ratios for high-performance lithium rechargeable batteries.

TPU combines the best properties of rubber and plastic, but has no plasticizers to leach out and cause allergic reactions or embrittlement over time. Thus, products made from polyurethane film & sheet, retain long-term flexibility and outstanding shelf life.

Thus, there is a need for an improved nerve stimuli regeneration system that may overcome one or more of the problems discussed above. In particular, there is a need for an improved nerve regeneration system that can efficiently optimize the treatment parameters, without requiring invasive exploratory techniques.

### OBJECTS AND SUMMARY OF THE INVENTION

The intrinsic transduction system of the present invention provides products, preferably an athletic shoe or athletic apparel, adapted to emit Piezoelectricity energy or information in response to impact. The product comprises a molded part having a Nerve stimulating unit or a midsole device at least partially molded therein. The unit comprises an impact-sensing element made from a polymeric piezoelectric material, a battery means, a stimulus device or an information display device and a circuit connected to said piezoelectric material. The stimulus unit is responsive to electrical energy produced upon impact which permits the nerve stimulus device to be energized from the battery or any information displaying device to be activated. In one embodiment, the electrical energy resulting from each impact is used as a trigger to operate nerve stimulating unit via the circuit incorporated therein, and the amount and/or duration of the stimulus emission can be independently determined/controlled by appropriate design of the circuit.

The product is preferably a spin disk, garment or shoe, particularly a prosthetic spinal disc, athletic garment or a sports shoe. The molded outsole part is preferably a thermoplastic unit structure, with at least a midsole, the circuit and the transducer stimulus device disposed in or molded into the midsole part of the structure, and with the piezoelectric stimulus-emitting device being arranged to emit energy outwardly from said mid part and/or by leads. The polymeric piezoelectric material may be molded into the midsole part of the structure, preferably in the region of maximum stress.

In still another embodiment, the circuit responds to the magnitude of the electrical energy produced by the piezoelectric material and thereby selectively energizes one or more of the nerve stimulating devices depending on the amount of electrical energy produced. In this manner, a visual indication of the magnitude of the pressure exerted upon the sole can be displayed.

The present invention provides a stimulating pulse having frequency components falling within predetermined frequency band limits. This pulse reliably elicits a touch response without the heretofore attendant noxious sensation mentioned above. It has been demonstrated that the differential excitation of the touch fibers relative to pain specific fibers inhibits the transmission of pain to the conscious centers. The type of stimulation specified herein, optimizes the differential excitation between touch and pain specific fibers, thus optimizing the inhibition of pain transmission.

### BRIEF DESCRIPTION OF THE DRAWINGS

FIG. 1 is a top view of the piezoelectric disk orb control of the present invention;

FIG. 2 is a side view of the piezoelectric disk orb control of the present invention;

FIG. 3 is a top view of the piezoelectric disk orb of the present invention;

FIG. 4 is a side view of the piezoelectric embed disk orb of the present invention with a base;

FIG. 5 is an elevation view of the piezoelectric disk battery capacitor of the present invention;

FIG. 6 is a top view of the piezoelectric disk control of the present invention;

FIG. 7 is a side view of the piezoelectric vertebral disc of the present invention;

FIG. 8 is a perspective view of the piezoelectric vertebral disc of another embodiment;

FIG. 9 is a side view of the mid-out sole vertebral disc of the present invention;

FIG. 10 is a side view of the battery capacitor vertebral disc of the present invention;

FIG. 11 is a perspective front view of therapeutic garments embodiment of the present invention.

FIG. 12 is a perspective rear view of therapeutic garments embodiment of the present invention.

FIG. 13 illustrate views of footwear product including features of the present invention;

FIG. 14 illustrate views of footwear product including features of the present invention;

FIG. 15 is an elevational perspective view of a cleat of the present invention;

FIG. 16 is a perspective top elevation view of shoe insole according to the present invention;

FIG. 17 -19 are side elevations view of shoe sole according to the present invention;

FIG. 20 is a block schematic circuitry diagram of the transducer according to the invention;

FIG. 21 is a schematic circuitry diagram of a negative ion transducer according to the invention.

### DETAILED DESCRIPTION OF A PREFERRED EMBODIMENT

Negative ions are odorless, tasteless, and invisible molecules that we inhale in abundance in certain environments. Think mountains, waterfalls, and beaches. Once they reach our bloodstream, negative ions are believed to produce biochemical reactions that increase levels of the mood chemical serotonin, helping to alleviate depression, relieve stress, and boost our daytime energy.

Ions are molecules that have gained or lost an electrical charge. They are created in nature as air molecules break apart due to sunlight, radiation, and moving air and water. You may have experienced the power of negative ions when you last set foot on the beach or walked beneath a waterfall. While part of the euphoria is simply being around these wondrous settings and away from the normal pressures of home and work, the air circulating in the mountains and the beach is said to contain tens of thousands of negative ions—Much more than the average home or office building, which contain dozens or hundreds, and many register a flat zero.

Thus, there is an increasing interest in external electrical skin stimulation for such purposes as pain suppression, neuro-muscular stimulation, communication systems, etcetera. Obviously, many modifications and variations of the present invention are possible in light of the teaching of devices shown in U.S. Pat. No. 1,059,090, U.S. Pat. No. 1,305,725 and U.S. Pat. No. 6,703,785. Specifically, there are many alternative ways of transducing the optimized waveforms disclosed herein which do not depart from the intended scope of the application. Accordingly, within the scope of the appended claims, the invention may be practiced otherwise than as specifically described.

### DETAILED DESCRIPTION OF THE INVENTION

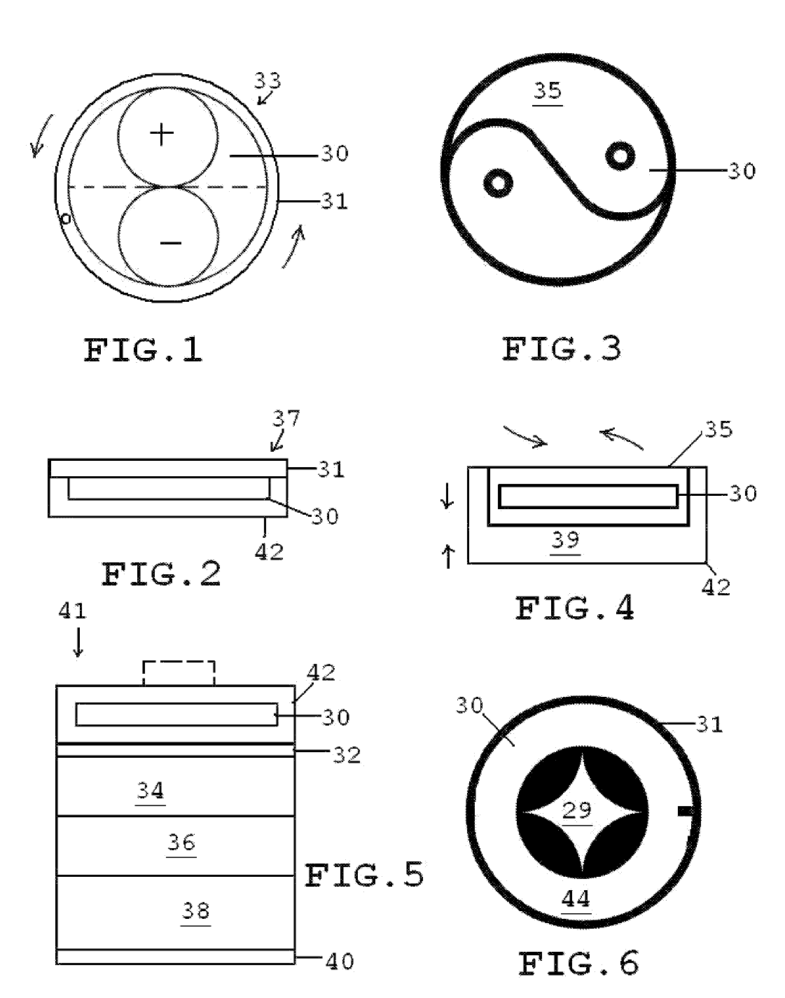

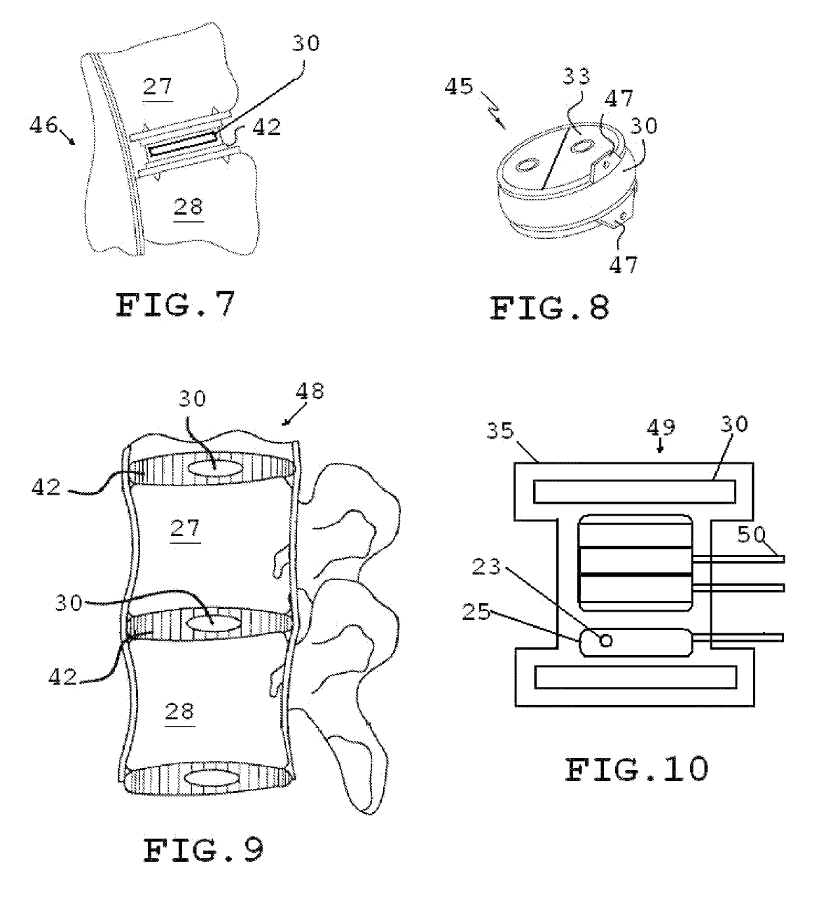

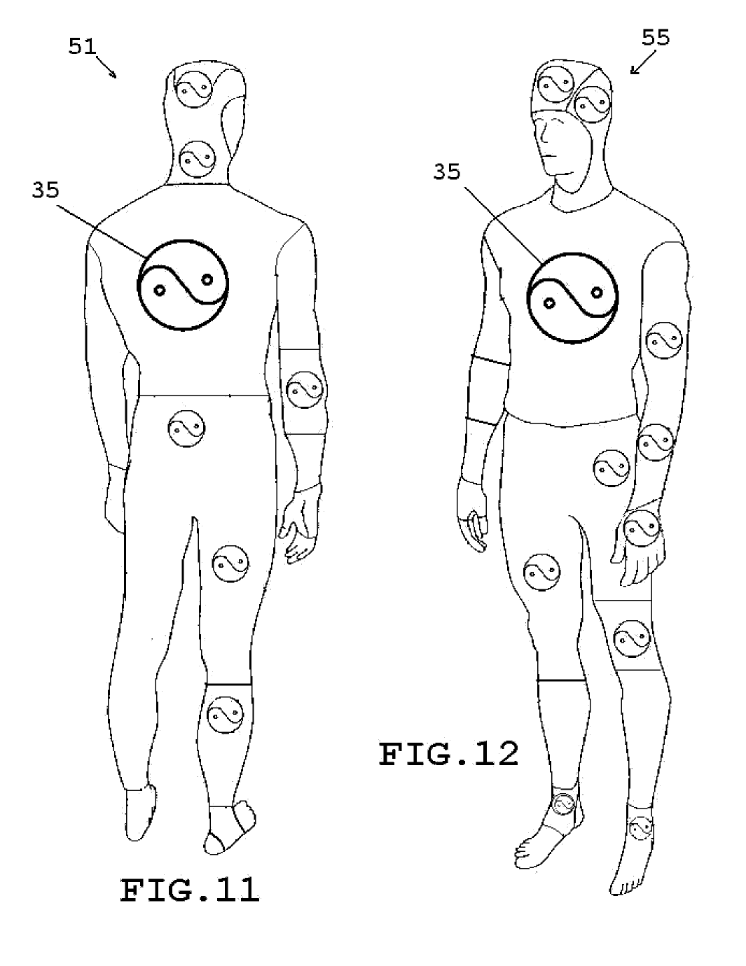

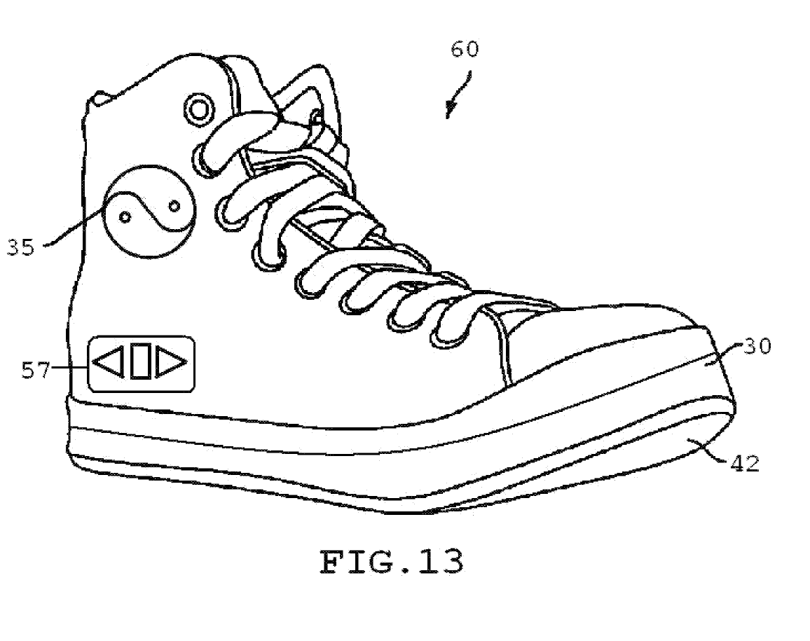

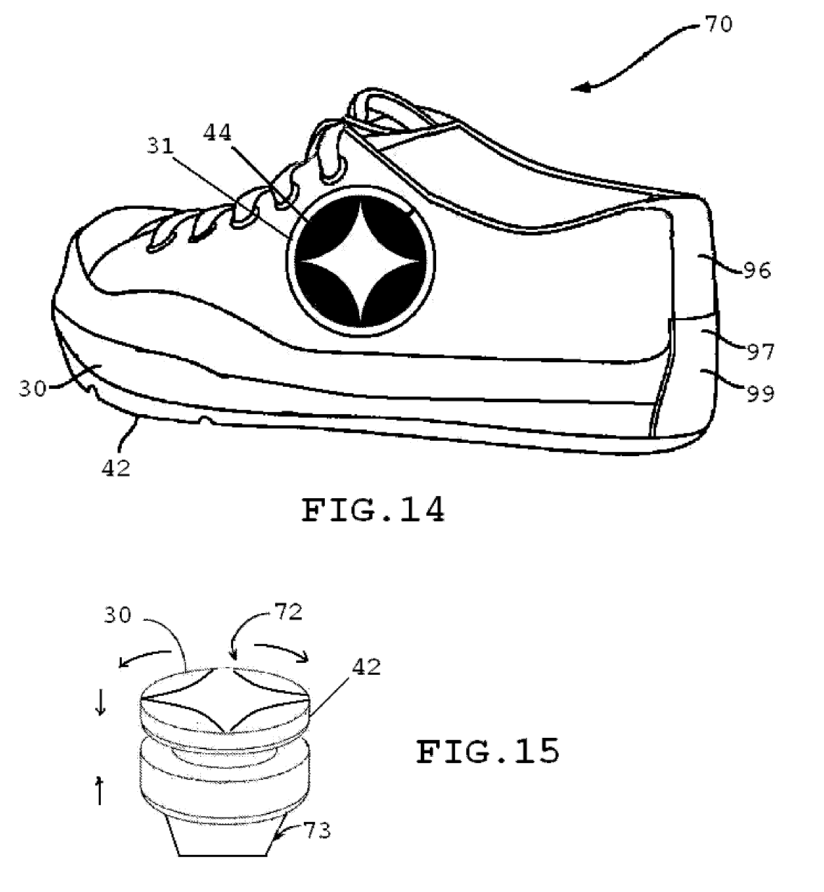

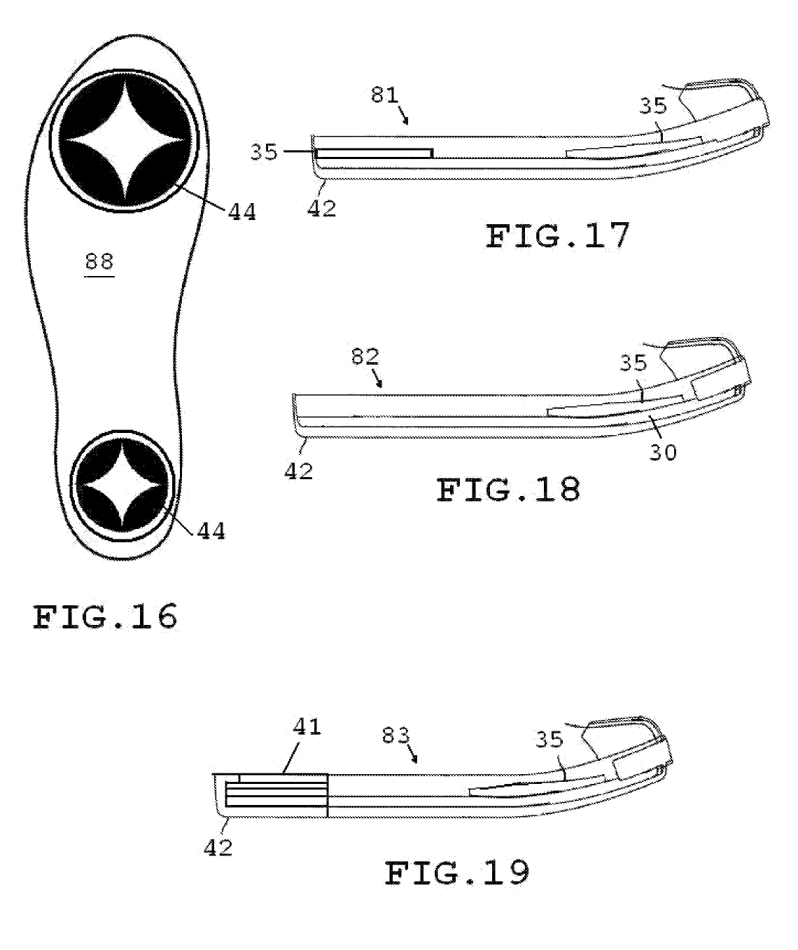

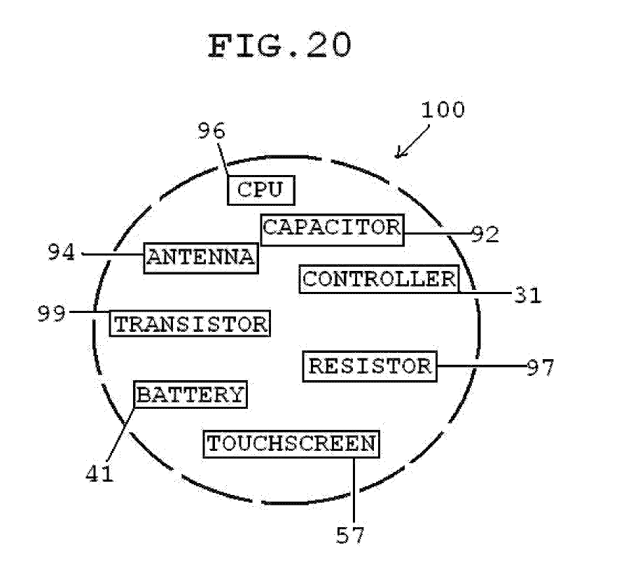

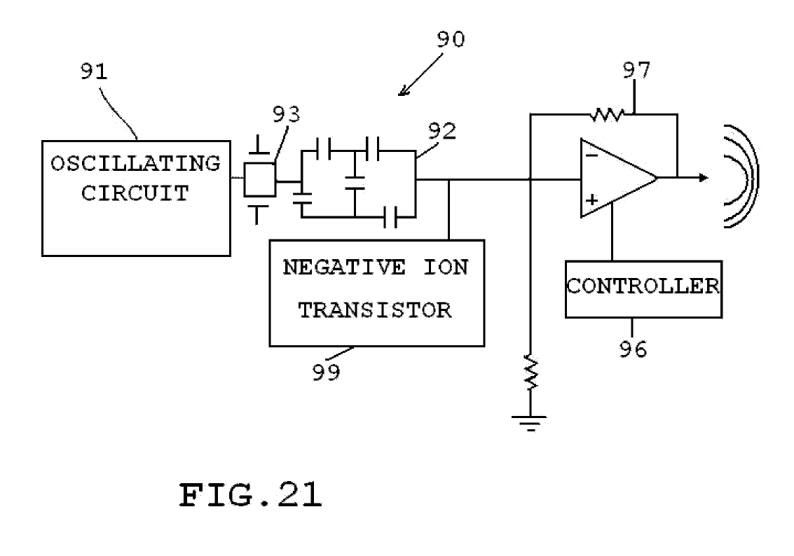

FIGS. 1, FIG. 2 and FIG. 3 show the construction of a spin Piezoelectric transducer 33, 35, 37, 39 according to the invention. The illustrated piezoelectric transducer FIG. 1 thru 3 is a module comprising a thin and small case disk housing outsole 42. FIG. 4 transducer has a piezoelectric insole 26 embed coupler outsole 42 base member accommodated about the midsole 30 casing. FIG. 5 battery transducer 41, starting from a regular lithium-ion coin battery, replaced the usual divider between electrodes with a polyvinylidene difluoride film whose piezoelectric nature produces a charging action inside that gap through just a little pressure. Further, modified (AL foil 32, LiCoO2 34, PVDF 36, TiO2 NT 38, Ti foil 40) with the attachment of a mid-out housing case. FIG. 5 & FIG. 6 an oscillating transducer 33,44 incorporates a controller 31 and designs 29 may form an abstract or geometrical pattern, emblem, or a logo or one or more alphanumeric characters constituting, for example, a trademark of the manufacturer. The ends of the fibers at these various points may be colored, e.g. with different colored translucent inks or dyes. FIG. 7 and FIG. 9 shows two adjacent vertebrae 27 and 28 with mid 30-out 42 molded transducer disc prosthesis 46,48 replacing the natural disc. Disc prosthesis 46 is a representation of the Charité prosthesis modified by the inclusion of out 42 plate transducers 35 and or mid fluid reserve 25, battery capacitor 41, transistors, antenna. FIG. 8 shows a Bryan similar cervical disc 45. Note that it is only recommended as a prosthesis for the cervical vertebrae. It has a mid 30 (not visible) between two molded out plates transducers 33 (only one shown). A flexible middisk 30 membrane between the molded outdisk 42 case housing surrounds the transducers 35 and or mid fluid reserve 25, battery capacitor 41, transistors nucleus. Applicant understands that current models of the cervical disc do not have the small tabs 47. FIG. 9 illustrates vertebral bodies 27 and 28 are separated by molded intervertebral mid 30-out 42 discs. Each disc has a nucleus midsole 30 surrounded by an annulus outsole 42. FIG. 10 represents a intervertebral prosthetic housing a battery capacitor 41 and end transducers 35. This embodiment may also house mid fluid 25 reserve accessible by a catheter aperture 23, battery capacitor 41, transistors and leads 50 to damaged nerve endings. FIG. 11 and FIG. 12 depict athletic apparel transducers head to toe garments according to embodiments of the present invention. Transducer 35 garment 51,55 may be adapted to be worn by a wearer therapeutically. The shoes 60,70 shown in FIG. 13 and FIG. 14 comprises a battery transducer 41, unitary sole-and-heel structure attached by molding or other means to a midsole 30. The transducers 35,44, forefoot sole and heel battery transducer 41 structure may be molded to the FIG. 16 insole 88 midsole 30 using methods well known in the art. Located or molded within the FIG. 17, FIG. 18, FIG. 19 sole 81,83 of the forefoot sole 81,82,83 and heel battery transducer 41 structure, preferably adjacent to a point of maximum stress (i.e. near the part corresponding to the ball or heel of the wearer's foot) is a piezoelectric 93 impact battery capacitor 41 comprised of a sheet or layer of polymeric piezoelectric material. This piezoelectric impact battery capacitor 41 preferably comprises polyvinylidene fluoride (PVDF) which has been stretch oriented and electrically polarized to enhance its piezoelectric properties. Such materials are known in the art. Referring to FIGS. 1 thru 19, the piezoelectric transducers 33, 35, 37, 39, 44 is electrically conductive to a FIG. 20 & FIG. 21 circuits 90,100 which contains a battery capacitor 41,92. Additional embodiments include but not limited to fluid 25 reserve, controllers 31,96, touchscreen 57, CPU 96, transistor 99 chips, antenna 94, resistor 97, oscillator 91 and leads 50. Said transducer parts communicate with design 29 emboss numerals, letters and emblems including logos, trademarks and fonts. FIG. 15 provides spark transducer mid 30 out 42 cleats 72 for athletic shoes, spark cleats 72 have a piezoelectric sole attachment member transducer disk 35 having a longitudinal axis for fitting into sole attachment means in the soles of the shoes and coupled traction edge 73. FIG. 13-14 and FIG. 16-19 detail various embodiments of the transducer 35 shoe mid 30-out 42 sole and or insole 88 including CPU 96, resistor 97 transistor 99 chips, controllers 31 and battery capacitors 41,92. FIG. 20 is a block circuit diagram showing the transducer, antenna 94, touchscreen 57, resistor 97, controller 96, transistor 99 and battery 98 capacitor 92. The transistor 99 has an oscillation 91 using a piezoelectric 93 transducer as oscillating means charges the battery 98 capacitor 92, transistor 99 boosts, controlled 96 and dispersed. FIG. 21 is a circuit diagram showing the negative ion transistor 99. The negative ion transistor 99 has an oscillating circuit 91 using a piezoelectric transducer as oscillating means. The oscillating circuit 91 generates a signal 26 at a frequency 101 of, for instance, 75 kHz as resonant frequency 101 of the piezoelectric 93 transducer 33, 35, 37, 39, 44 (which is determined by the length direction dimension) or the neighborhood (±5 kHz) of the resonant frequency 101.

### ALTERNATIVE EMBODIMENTS

Alternatively, send out signals to muscle synapses where chips work with common signaling substances, for example acetylcholine. According to yet another aspect, the FIG. 21 system may comprise a first lead 50 may configured to deliver an electrical signal, and a second lead 50 configured to deliver the therapeutic fluid 25 to the damaged nerve. The lead may comprise a catheter tube lead 50 in fluid communication with the fluid delivery device and configured to deliver the therapeutic fluid to the damaged nerve. According to still another aspect, the control module may comprise a fluid delivery device configured to provide a therapeutic fluid to the damaged nerve. For example, the control module comprises a fluid port for supplying fluid to the fluid delivery device. All embodiments may be produced with alternative materials, in the art.

I claim:

1. An intrinsic transducer system method, comprising: athletic apparel, shoes, prosthetic disc and spark cleats wherein the piezoelectric stimulation comprises a therapeutic electric signal:
a. method for constructing a an athletic shoe comprising sole, midsole, outsole, and bladders, having the appearance of a novel athletic shoe; and wherein said transducer assembly of parts, said parts communicate design with emboss numerals, letters and emblems including team logos, trademarks and fonts communicate with emboss wherein oscillating transducer means incorporated within the sole comprising piezoelectric material for generating impact signals upon application of pressure to the material, the sole imparting pressure to the material when impressed against a surface; monstable multivibrator circuit means interconnecting the pressure to battery capacitor transducer means, the ion and the power means, wherein the circuit means responds to the impact to control the power means to power the ion in response to the pressure imparted by the sole on the material; wherein the circuit means comprises; input transistor means for enhancing the frequency from the pressure transducer means and emitting the piezoelectricity to the circuit means, the input means including sensitivity resistor control means across which the ions are applied to stimulate nerves and tissue; and
b. a transducer spark cleat for an athletic shoe, which cleat comprises a sole attachment member having a longitudinal axis for mounting the cleat to the shoe; wherein a hub having a planar upper portion perpendicular to the attachment member having a first periphery and a rounded lower portion having a second periphery; and
c. an athletic apparel negative ion transducer garment, comprising: a textile portion; a transducer device retention element coupled by bonding to the textile portion; and wherein said oscillating transducer assembly of parts, said parts communicate with emboss numerals, letters and emblems including team logos, trademarks and fonts; and
d. a negative ion transduce athletic apparel garment, piezoelectric support structures for foot-receiving devices as midsole and/or outsole structures for articles of footwear including an impact-attenuating material, cleat for athletic shoes which provides a sole attachment member; a hub having a planar upper portion for contacting the shoe sole, a rounded lower portion for bearing weight of the user, and an edge; and resilient traction elements, wherein prosthetic systems and methods for promoting nerve regeneration are disclosed in exemplary embodiments, a nerve regeneration system may include a lead configured to be placed in a body proximate a damaged nerve, a portion of the lead being configured to stimulate the damaged nerve.

2. A method for constructing a transducer prosthetic vertebrae disc sole comprising construction of an athletic shoe midsole, outsole, and bladders, having the appearance of a novel vertebrae disc:
a. prosthetic transducer implant configured for placement between opposing bones that apply pressure to the implant during articulation, the implant comprising: a battery capacitor, transistor, resistor, antenna, CPU, a fluid-filled reservoir; and a molded body coupled to at least one of the bones and the reservoir to provide cushioning during articulation; and
b. negative ion transducer adapted for use as a stimulation for the purpose of organic pain suppression which comprises: wherein the parameter of the stimulation comprises a parameter associated with the electric signal; wherein the parameter comprises one or more of strength, direction, current, or voltage of the electric signal; further comprising a lead coupled and configured to deliver electric stimulation to the damaged nerve; wherein the control module is enclosed in a substantially sealed housing and the lead extends from the housing; wherein multiple leads extend from the housing. output leads for transmitting a signal generated by the circuit means to the tissue and nerves, including a first resistive means for limiting the current through the leads; and
c. transducer nerve regeneration system comprising: a lead configured to be placed in a body proximate a damaged nerve, a portion of the lead being configured to stimulate the damaged nerve; and a control module configured to a signal indicative of the nerve's response to the stimulation and adjust a parameter of the stimulation in response to the monitored signal; and
d. the system of claim 2, wherein the stimulation comprises a therapeutic electric signal.
e. The system of claim 2, wherein the fluid delivery system comprises a reservoir for storing fluid associated with the fluid delivery lead.

3. A battery transduction oscillation system method of charging or enhancement where piezoelectric transducer and/or battery disks form a modified version of capacitor, wherein said capacitor(s) having an outside housing and wherein the improvement comprising an outside diameter or linear distance constitute and wherein said battery capacitor is a sub-system of an nerve regenerator, sub-system of an athletic apparel, sub-system of a vertebrae disc prosthetic, sub-system of a shoe, sub-system of a cleat, sub-system of a negative ion system, and:
a. negative ion transducer system method, comprising: A lithium battery, A negative ion transducer for generating negative ions by piezoelectric oscillation, said negative ion transducer circuit comprising: a negative ion transistor operable to output a DC high voltage; a resistor circuit operable to generate a negative high voltage from the DC high voltage from said negative transistor; a discharge adapted to emit electrons; an oscillating circuit operable to generate a signal having a frequency in a neighborhood of a resonant frequency of said negative transistor for controlling a frequency of an DC voltage.
